(19) United States
(12) Patent Application Publication
(10) Pub. No.: US 2003/0211065 A1
(43) Pub. Date: Nov. 13, 2003

(54) Methods and compositions for diagnosing and treating hypotrichosis simplex

(76) Inventors: Elon Pras, Ramat Efal (IL); Etgar Levy-Nissenbaum, Tel-Aviv (IL); Moshe Frydman, Petah-Tiqwa (IL); Theodore Thederahn, Los Angeles, CA (US)

Correspondence Address: Laurie A. Axford, Morrison & Foerster LLP, Suite 500, 3811 Valley Centre Drive, San Diego, CA, 92130, US

(21) Appl. No.: 10/316,175

(22) Filed: Dec. 9, 2002

Related U.S. Application Data
(60) Provisional application No. 60/338,188, filed on Dec. 7, 2001.

(51) Int. Cl. A61K 7/06; A61K 31/70; A61K 7/11; A61K 48/00; A61K 38/16

(52) U.S. Cl. 424/70.13; 424/70.14; 514/2; 514/44

(57) ABSTRACT

This invention relates to the identification of genes linked to hair loss and the retardation of hair growth. In one embodiment, the invention relates to the identification of a gene, a mutation of which plays a role in the onset of a nonsyndromic alopecia, such as hytrichosis simplex of the scalp (HSS). More particularly, it relates to the corneodesmosin (CDSN) gene, which encodes the protein known as corneodesmosin.

This application claims priority under 35 U.S.C. 119(e) to provisional application No. 60/338,188 filed Dec. 7, 2001, the contents of which are incorporated herein by reference.

### FIELD OF THE INVENTION

This invention relates to the identification of genes linked to hair loss and the retardation of hair growth. In one embodiment, the invention relates to the identification of a gene, a mutation of which plays a role in the onset of a nonsyndromic alopecia, such as hytrichosis simplex of the scalp (HSS). More particularly, it relates to the corneodesmosin (CDSN) gene, which encodes the protein known as corneodesmosin.

### BACKGROUND OF THE INVENTION

Alopecia generally refers to a loss of hair. Common causes of alopecia include, e.g., autoimmune disorders, hormonal imbalances, neurological conditions and genetic abnormalities. One form of alopecia is hypotrichosis simplex, which is a condition that can affect all body hair (“generalized form”) or can be limited to affecting the scalp. In this later case, i.e., hypotrichosis simplex of the scalp, it is conveniently refereed to as “HSS.”

HSS is a rare isolated alopecia with autosomal dominant inheritance and full penetrance. Men and women are equally affected. Usually, patients with the scalp-limited form present with normal hair at birth and in the first years of life. They experience a progressive, gradual loss of scalp hair beginning at the middle of the first decade leading to almost complete loss of scalp hair by the third decade. A few sparse, fine, short hairs can remain in some individuals. The body hair, beard, eyebrows, axillary hair, teeth, and nails are normally developed. Morphological examination of hairs by light and scanning electron microscopy shows no gross abnormality of hair shaft, although hairs from patients with advanced hypotrichosis show focal areas of defective cuticular structure. Usually, no scarring or inflammatory changes are present.

Toribio, et al., Brit. J. Derm 91:687-696 (1974), incorporated by reference, first described HSS, via observation of a large Spanish kindred, which exhibited afflicted individuals in 8 generations. Affected children were normal at birth, but began to exhibit hair growth retardation at ages 5-12. By the time the subjects were 20-25 years old, all affected subjected reached a stage where only a few sparse hairs were observed on the scalp. No other hair related abnormalities were observed.

Hess, et al Am. J. Med. Genet. 39:125-129 (1991), describes afflicted individuals over six generations in a Caucasian family, including one example of male-to-male transmission. The family studies resembled the extended family of Toribio, et al, supra, except HSS was fully manifest at birth, and progressed. Ibsen et al., Aeta Derm. Veneral 71:349-351 (1991) also report a multigeneration family where HSS onset was observed at ages 6-17, with almost total scalp alopecia at ages 14-21. No associated ectodermal defects were found, however.

Zhou, et al, Proc. Natl. Acad. Sci. 92:9470-9474 (1993), incorporated by reference, describe a gene approximately 160 kilobases telomeric to HLA-C. The gene was only expressed in skin, and was thus designated the “S” gene. In situ hybridization showed the S gene (which encodes the CDNS gene) expression being restricted to differentiating keratinocytes in the granular layer of the epidermis. The gene was found, via Northern blotting, to be expressed as 2.2 and 2.6 kb mRNAs. Further analysis showed that the gene contains two exons, and predicts a 486 amino acid protein as translation product. The protein includes a 16 amino acid signal sequence. The protein shows some homology to lorelin, keratin-1, and keratin-10, all of which are major components of the granular layer. The S gene and its protein product are shown in FIGS. 2A and 2B, respectively.

Betz, et al, describe genomic wide linkage analysis in two families, and localize the condition to chromosome 6p21.3 (Betz, et al Am. J. Hum. Genet. 66:1979-1983 (2000)). Mapping was confirmed via analysis of the family described by Toribio, et al., supra. Combined haplotype data identified a critical, 14.9 cM interval between markers D6S276 and D6S 1607. However, as reported therein, “there is no clear candidate gene for the disease” located within this interval (at page 1982).

A large multi-generation Israeli family of Jewish-Yemenite origin, previously described by Kohn and Metzkeru (Clin. Genet. 31: 120-124 (1987)), showed linkage to the same region (unpublished data). By identifying new polymorphic markers within the interval and genotyping family members from the Israeli family, the critical region was reduced to 9.5 Mb, between clones Z85996 and AL133255. Focusing on genes from this interval expressed in skin, we found mutations in the corneodesmosin (CDSN) gene, which encodes the protein referred to as corneodesmosin.

The CDSN gene, which is shown in FIG. 2C, is composed of two exons, and encodes a protein of 529 amino acids as shown in FIG. 2D (Guerrin, M. et al., J. Biol. Chem. 273: 22640-22647 (1998)). CDSN is a highly polymorphic gene. Several missense substitutions and two trinucleotide deletions/insertions have been described as common variants in the general population. (Guerrin, M. et al. Identification of six novel polymorphisms in the human corneodesmosin gene. Tissue Antigens 57, 32-38 (2001)).

The CDSN protein, shown in FIG. 2E, is expressed in cornified squamus epithelia. It is so named because of its association with corneodesmosomes. These are intracellular structures which are involved in desquamation, i.e., the shedding of superficial corneocytes from skin surfaces. Corneodesmosomes form part of the cell-to-cell adhesive network that keeps the cornified layer cohesive. Corneocytes are enucleated cells which derive from keratinocytes during late stages of terminal differential of cornified squamous epithelia, such as the epidermis.

Simon et al., identified the protein immunologically, and characterized it as a 52-56 kDa glycoprotein, but as it progresses to the upper cornified layer, it is proteolytically digested into smaller molecules (Simon et al., J. Biol. Chem., 272:31770-31776 (1997)). The reduction in its size seems to account for the loss of adhesivity of the cornified cells finally resulting in desquamation. CDSN is also expressed in the three epithelial components of the inner root sheath of hair follicles where it is probably also associated with corneodesmosomes and involved, like in the epidermis, in cell cohesion.

CDSN is known to be synthesized during the differentiation of corneocytes, and is incorporated into desmosomes just before the structural modifications which result in transformation of these cells into corneodesmosomes. CDSN is covalently linked to cornified envelope, and is a key molecular component of the protein system responsible for intracellular cohesion within the horny layer. It is a highly conserved protein, found in all mammalian systems studied. It has been found to be overexpressed in many hyperkeratotic human cutaneous diseases, including winter xerosis, psoriasis, and various ichthyoses. For example, it has been suggested that CDSN is implicated in the impaired desquamation that is characteristic of psoriasis. See Ahnini, et al., Hum. Molec. Genet. 8:1135-1140 (1999); Asurmalahti, et al, Hum. Molec. Genet 9:1533-1542 (2000), and PCT WO 01/62788.

Accordingly, the CDSN gene is an exemplary diagnostic and therapeutic target for HSS. In particular, mutations in this gene have now been identified and linked to HSS. Using the CDSN gene as a model system, other genes that are implicated in nonsyndromic alopecia can also be identified and targeted in diagnostic and therapeutic applications.

### SUMMARY OF THE INVENTION

The present invention relates to the identification of genes that play a role in the onset of nonsyndromic alopecia. In one embodiment, it relates to the discovery that corneodesmosin plays a role in hair growth, and abnormalities of the corneodesmosin gene are associated with hypotrichosis simplex of the scalp (HSS).

Accordingly, one aspect of the present invention relates to therapeutic compositions comprising an effector of a corneodesmosin gene function associated with hair growth in a pharmaceutically acceptable carrier, wherein said effector targets a nucleic acid sequence encoding a hair growth-associated corneodesmosin activity. The effector can be, e.g., a small molecule, a nucleic acid or a protein. In addition, the hair growth-associated corneodesmosin activity can be, e.g., cell adhesion or signal transduction.

In another aspect, the present invention relates to a therapeutic composition comprising an effector of corneodesmosin protein activity associated with hair growth in a pharmaceutically acceptable carrier, wherein said effector targets a subregion of the corneodesmosin protein exhibiting a hair growth-associated corneodesmosin activity, such as corneodesmosin protein proteolysis. In one embodiment, the effector may be a peptide that mimics a corneodesmosin proteolysis fragment.

In yet another aspect, the present invention relates to a method for modulating hair growth comprising administering to a subject an effective amount of a therapeutic agent in a pharmaceutically acceptable carrier, wherein the agent modulates at least one corneodesmosin activity associated with hair growth, which may either inhibit or promote hair growth.

A further aspect of the present invention is a method for screening an agent for hair growth modulation activity comprising the steps of: incubating cells transfected with an expression construct capable of expressing corneodesmosin protein with or without the agent; and comparing activity of the corneodesmosin protein from the cells incubated with or without the agent, wherein the activity is associated with hair growth, and may be, e.g., cell adhesion or signal transduction.

The present invention also includes a method of diagnosing corneodesmosin gene-mediated alopecia or a propensity to develop said alopecia in a subject, comprising the steps of: determining a subregion of the corneodesmosin gene suspected of having a mutation that modulates corneodesmosin activity; preparing a nucleic acid probe that binds to the subregion; and performing a hybridization assay with the nucleic acid probe to detect the presence of the mutation. The subregion may encode a glycine-rich domain or a signal transducing domain. In addition, the diagnostic method may involve use of a probe specific for the premature stop codon in the corneodesmosin genes which is described in Example 1.

A method for treating alopecia is another aspect of the present invention which comprises administering a therapeutic agent in a pharmaceutically acceptable carrier, wherein the therapeutic agent comprises corneodesmosin protein or a fragment thereof.

Yet another aspect of the present invention is a method for treating alopecia comprising administering a nucleic acid construct to a subject comprising a promoter operably linked to a corneodesmosin gene, wherein said nucleic acid construct is capable of expressing corneodesmosin protein or fragments thereof after administration.

Also included within the present invention is a method for identifying a genetic target associated with genetic hair loss from a population including members exhibiting hair loss comprising the steps of: identifying the population; screening the population to locate a chromosomal region associated with the hair loss; comparing the chromosomal region to known genes expressed in skin located within the region; and rescreening the population to locate a genetic abnormality in the genes expressed in skin located within the region.

Other aspects of the present invention are described throughout the specification.

### BRIEF DESCRIPTION OF THE DRAWINGS

FIG. 1A depicts a schematic map of chromosome 6 showing the region of linkage, flanking clones, and genomic organization of the CDSN gene. The solid bars indicate exons, and the striped bars indicate the untranslated regions (UTRs).

FIG. 1B depicts the sequential proteolysis of the CDSN protein from a 52-56 kDa protein to a 15 kDa, and also shows release of fragments a-d during each of the three proteolysis steps. As depicted, the wavy lines represent the glycine-rich domains and the dark circle represents the N-glycosylation site. Simon, et al., J. Biol. Chem., 276:20292-20299 (2001). The cleavage fragments a, b, c, and d, are also shown.

FIG. 2A depicts the sequence of the Human S Protein mRNA (SEQ ID NO.11) and FIG. 2B depicts the sequence of the S-protein (SEQ ID NO.12), both from Zhou, Y. and Chaplin, D., Proc. Natl. Acad. Sci. U.S.A. 90(20):9470-9474 (1993)(GenBank AAA21321.1).

FIG. 2C depicts a partial sequence of the MHC region on chromosome 6p21.3 from nucleotide 6961-11700 (SEQ ID NO.13), which contains the corneodesmosin gene (GenBank Accession No. AC006163). The two exons (shorter exon 1, SEQ ID NO.67 and longer exon 2, SEQ ID NO.68) are shown in underlined text. The region encoding the corneodesmosin protein (FIG. 2D) is indicated with capitalized letters. The start codon, which is at position 15-17 in the corneodesmosin gene is indicated by an arrow. This also corresponds to GenBank AB023060 (exons 31143949-31144047 and 31139519-31141144).

FIG. 2D depicts the corneodesmosin mRNA (SEQ ID NO.14) (AF030130).

FIG. 2E depicts the sequence of the Human CDSN protein from Guerrin, M., et al., J. Biol. Chem., 273(35):22640-22647 (1998)(SEQ ID NO.15)(GenBank AAC24196; XP_{—}004564). The relationship between the sequences in FIGS. 2B and 2E is indicated by a single unmarked arrow.

### DETAILED DESCRIPTION OF THE INVENTION

This invention relates to the identification of genes that play a role in the onset of nonsyndromic alopecia, such as hypotrichosis simplex of the scalp (HSS). In an exemplary embodiment, the invention relates to the identification of a gene linked to hair loss and retardation of hair growth. More particularly, it relates to the corneodesmosin (CDSN) gene, which encodes the protein known as corneodesmosin.

Corneodesmosin Gene-Related Alopecia

Using the methods described herein, abnormalities in the corneodesmosin gene have been newly implicated as a cause of HSS. Accordingly, corneodesmosin provides an ideal target for new diagnostic and therapeutic methodologies related to alopecia that effectuate a modulation of normal corneodesmosin production and/or function. In addition, corneodesmosin activity may be modulated under appropriate conditions to inhibit hair growth.

Although not wishing to be bound by any particular theory, CDSN may play a role as a transducer of mechanical stress or “volume sensor” during the growth of hair follicles on the scalp. The two primary types of hair present in humans are the vellus hairs, common on most parts of the body and present in the “bald” regions of male pattern baldness, and terminal hairs, present on the scalp. Overall morphology and histological examination reveal no differences between the two types, except for their relative size. This has led to the suggestion that during follicle development, a chemical signal is produced either by the surrounding mesenchyme or within the developing follicle itself, that halts vellus follicle development in a “miniaturized” form. The source of this signal is unknown, but it is clear that a mechanical “volume” sensor, responding to increasing pressure from surrounding tissue as the follicle expands in size during development would be one possible solution to this problem. Since CDSN is present near the interface of the follicle with the surrounding mesenchyme (the inner root sheath) and around the dermal papilla which is suggested to be the location of the volume sensor, and since it shares many characteristics with known adhesion molecule based stress sensors, it is hypothesized that one of CDSN's activities is as a volume sensor. In addition, other corneodesmosomal proteins such as plakoglobin and desmoglein have been shown to modulate cell responses to mechanical stress.

Several mechanisms of signal initiation or transduction suggest themselves. First, as CDSN is progressively proteolyzed, it may lose interactions with adjacent extracellular matrix proteins which are then activated, initiating a signal that is then transduced to the cytoplasm. Second, one of the proteolyzed fragments of CDSN released after cleavage may have a cryptic binding site which is revealed by proteolysis and then able to bind to its cognate binding site thus initiating a signal. Finally, the loss of proteolyzed fragments may reveal a cryptic binding or phosphorylation site on CDSN that is then involved in initiating a transduced signal. A prematurely truncated CDSN may therefore be unable to interact with all of its normal signaling partners. Thus, a “short circuit” in follicular development is created at the vellus stage. Essentially, the volume sensor registers “follicle complete” before it has achieved its full size.

Cell Adhesion Molecules and Signal Transduction in Hair Growth

Recent evidence has clearly demonstrated that the specialized intercellular junctions in epidermal tissue modulate many other functions besides simple adhesion. During morphogenesis, for example, the growth of a hair follicle within the epidermis, mechanical forces generated by the dynamic rearrangements of cell-cell contacts are transduced via signaling molecules to the cytoskeleton which modulates changes in cell shape and motility that transform uniform sheets of cells into specialized three-dimensional structures (Hogan, Cell 96:225-233 (1999)). Much work has been done to demonstrate the critical importance of adherents junctions and their linkage to actin filaments via the resident adapter protein β-catenin in the morphogenesis of hair follicles (DasGupta and Fuchs, Development 126:4557-4568 (I 999)).

In a similar fashion, the cadherin family of calcium-dependent adhesion molecules that are the core of desmosomes are linked indirectly to intermediate filaments (IF) through adapter molecules (Koch and Franke, Curr. Opin. Cell Biol. 6:682-687 (1994)) and have been shown to respond to growth factors (Savagner et al., J. Cell Biol. 137:1403-1419 (1997)) and to associate with kinases and phosphatases (Fuchs et al., J. Biol. Chem. 271:16712-16719 (1996)). An important cell-biological window on the role of desmosomes as sensors has been provided by study of the epithelial blistering disease pemphigus vulgaris. Binding of autoantibodies directed against desmoglein 3 (Dsg3) have been shown to cause phosphorylation of Dsg3 leading to a dissociation from its partner plakoglobin thus causing a subsequent loss of cell-cell adhesion and the characteristic epithelial blistering. Transgenic mice without Dsg3 (Dsg3−/−) not only develop the characteristic skin lesions associated with pemphigus vulgaris, but also show hair loss during the telogen phase of the hair growth cycle. Histology revealed incomplete desmosomes and a loss of cell-cell adhesion in the outer root sheath epithelium (Koch et al., J. Cell Sci. 111:2529-2537(1998)).

Additionally, mice expressing the balding (bal) mutation have alopecia and have been shown to have a defect in adhesion molecules, leading to a separation of the outer root sheath from the inner root sheath in their hair follicles. It was demonstrated that this mutation (bal(J)) produced a premature stop codon in the transcription of the Dsg3 gene (Montagutelli et al, J. Invest. Dermol. 109:324-328 (1997)). Plakoglobin, the primary cadherin in desmosomes, whose ability to stimulate the lymphoid enhancer-binding factor (LEF/TCF) and its role in axis duplication (Merriam et al., Dev. Biol. 185:67-81 (1997)) both suggest mechanistic similarities to β-catenin, has been shown to be able to reduce the growth of hair follicles in transgenic mice (Charpentier et al., J. Cell. Biol. 149:503-520 (2000)), transform cells by specifically activating c-myc (Kolligs et al., Genes Dev. 14:1319-1331 (2000)) and can inhibit apoptosis by inducing Bcl-2 (Hakimelahi et al., J. Biol. Chem. 275:10905-10911 (2000)). These data suggest a critical sensory role for desmosomes in the turnover of junctional complexes and the remodeling of desmosomes during cycling and differentiation.

Finally, in a broader sense, evidence of direct activation of signal transduction pathways by desmosomes and other anchoring junctions with cadherin molecules continues to accumulate. N-cadherin can activate FGFr dependent neurite outgrowth in tumor cells, N— and E-cadherin can stimulate divergent differentiation pathways for embryonic stem cells, E- and VE-cadherin activate the phosphatidylinositol-3-(OH)-kinase dependent serine kinase PKB/Akt and contact inhibition by increasing levels of the cell-cycle dependent kinase inhibitor p27 (Anastasiadis and Reynolds, J. Cell Sci. 113:1319-1334 (2000)).

Diagnostic and Therapeutic Targets in the Corneodesmosin Gene and Protein

The present invention relates to targeting of the corneodesmosin gene and/or protein in the diagnostics and therapy of alopecia and other conditions relating to hair growth. Such diagnostics and therapeutics can target any portion of the corneodesmosin gene or protein, either directly or indirectly. This intends that the agent may interact directly with the gene or protein. An example of direct interaction is a nucleic acid-based agent hybridizing with the corneodesmosin gene, or a protease-based agent cleaving the corneodesmosin protein. An example of an indirect interaction is a small molecule inhibiting the functioning of a corneodesmosin-cleaving protease or a nucleic acid hybridizing and thus modulating the activity of a corneodesmosin gene-controlling regulatory sequence. In any event, the word “agent” is used herein to refer to a chemical moiety or molecule, including without limitation a small (less than 1000 mol. wt.) molecule, a nucleic acid (which also includes polynucleotide fragments), or a protein (which also includes peptide fragments) or which modulates corneodesmosin activity. Such agents can also be referred to as “effectors” of corneodesmosin activity associated with hair growth, which includes agents that either inhibit or promote hair growth via their effect on a hair growth-related activity of corneodesmosin, such as signal transduction or adhesion.

Other examples of modes of actions of agents include, inter alia, small molecules that inhibit proteolysis, those that bind to a cryptic binding site or cryptic modification site once revealed or those that interfere with a required interaction with another extracellular matrix protein.

Accordingly, the agent may target any portion of the CDSN gene or protein. However, in some embodiments, the agent is designed to target only a portion, subregion, domain or motif (collectively “subregions”) of the gene or protein associated with a particular corneodesmosin-associated function or activity thereof that is associated with growth. No previous role for CDSN has been postulated in hair growth. Evidence that CDSN's molecular partners in corneodesmosomes have direct or indirect ability to initiate signal transduction pathways, which is presented herein, suggests that CDSN, either alone or through its ability to affect other desmosomal proteins may have similar abilities. Examination of CDSN's sequence reveals several interesting homologies, and the identification of several particular subregions are described below.

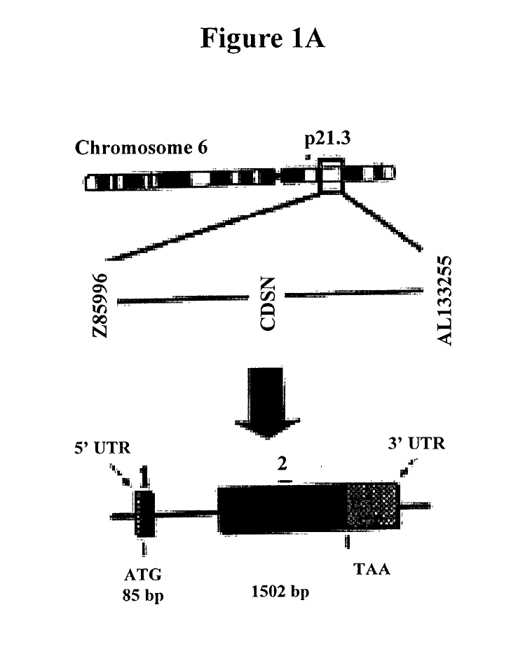

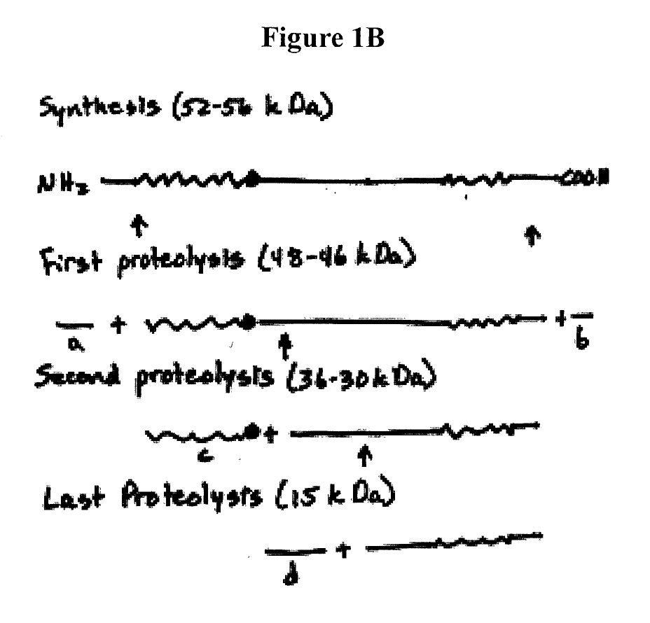

Human CDSN is specific to the cornified epithelium and the inner root sheath of hair follicles (Haftek et al., J. Histochem. Cytochem. 39:1531-1538 (1991)) and is thought to be involved in cell-cell adhesion via both homophilic and heterophilic interactions of its glycine loop domains (Jonca et al., J. Biol. Chem. 277:5024-5029 (2002)). Furthermore, during the maturation of the stratum corneum, this protein is progressively proteolyzed, first from its full-length 52-56 kDa form, to a 46-48 kDa fragment lacking portions of both the C-terminal and N-terminal regions. A second proteolysis step removes further portions of the N-terminal region to form a 30-36 kDa form, and a final proteolytic cleavage leaves a 15 kDa core fragment which is present only at the surface of non-cohesive corneocytes (Simon et al. (2001) and Simon et al., (1997), supra). See FIG. 1B.

Accordingly, corneodesmosin activity can be modulated by agents that affect corneodesmosin proteolysis. Such affects can easily be determined using known methods. For example, keratinocytes known to express corneodesmosin can be cultured and corneodesmosin expression and proteolysis can be studied using any of a number of known biochemical methods, such as antibody-based assays specific for corneodesmosin domains, gel electrophoresis to study corneodesmosin cleavage under different conditions, etc.

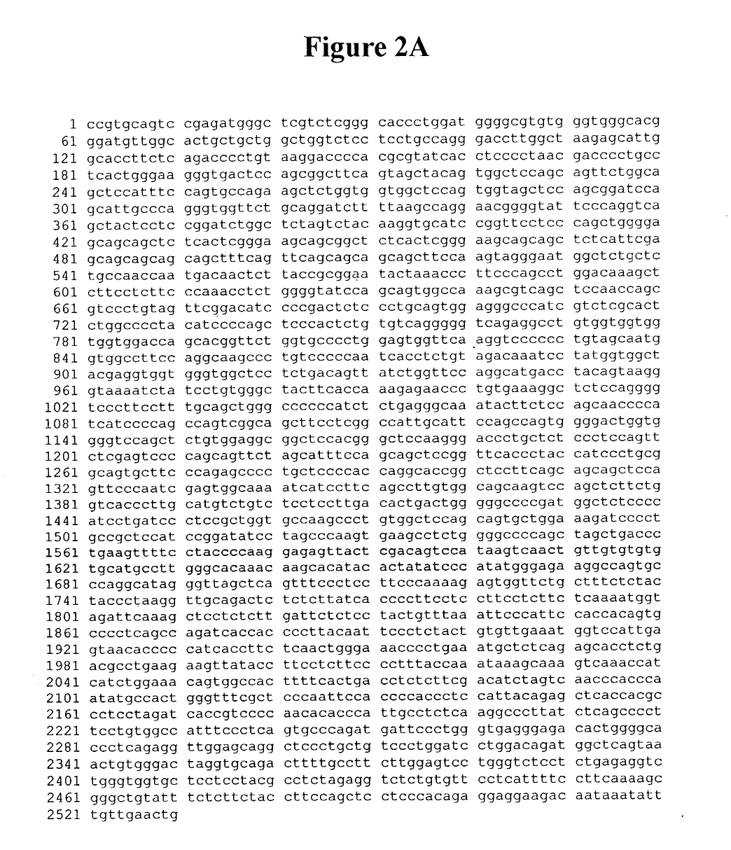

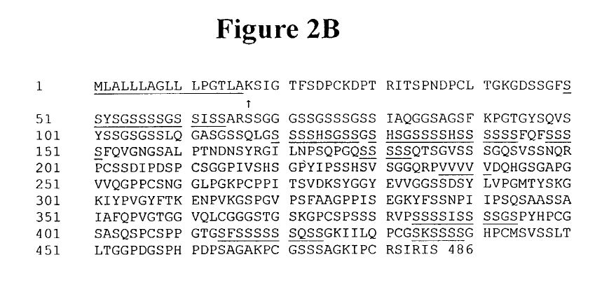

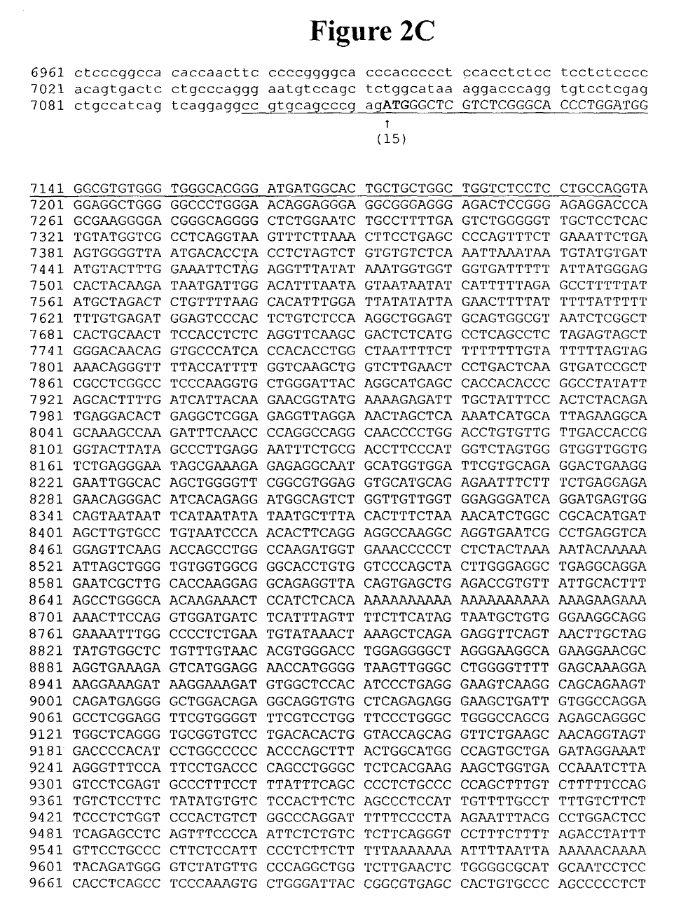

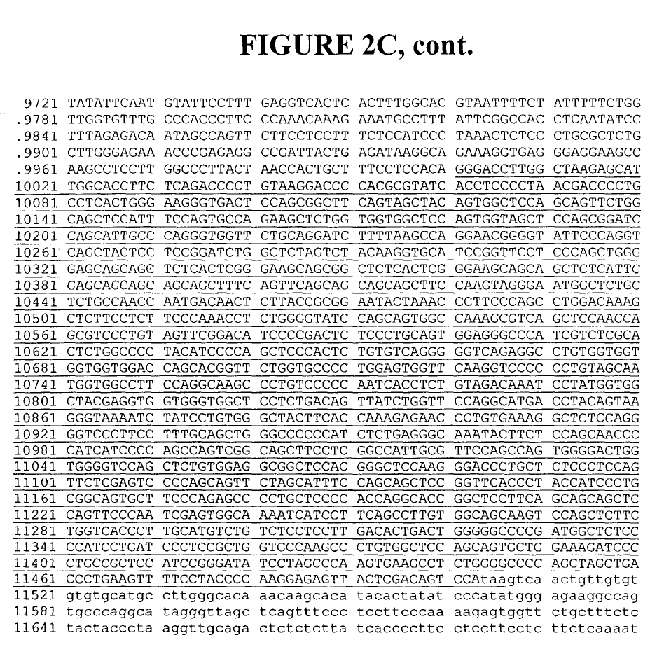

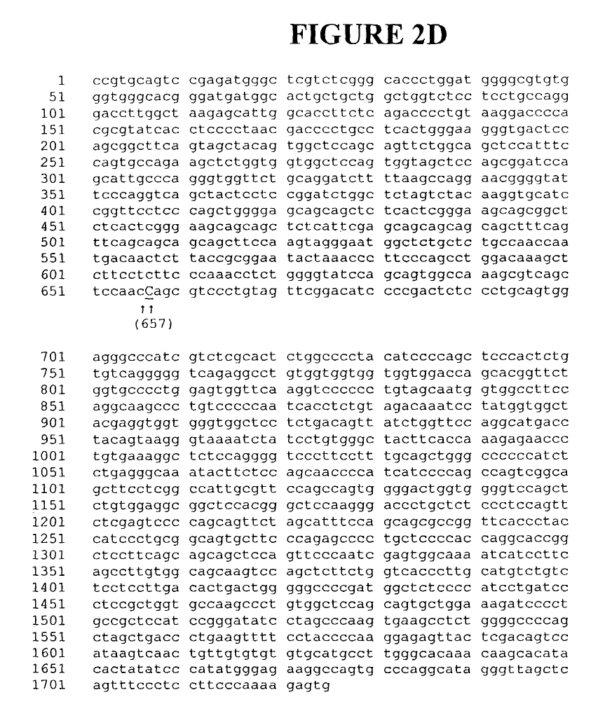

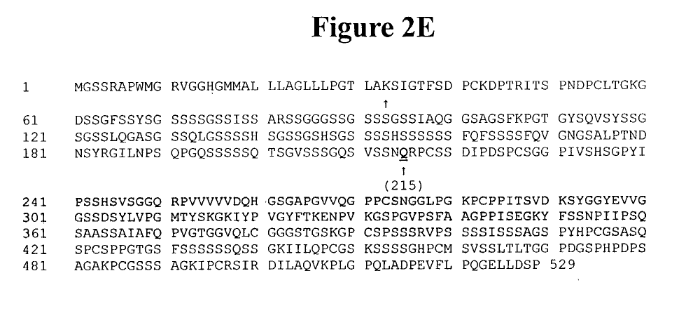

Simon et al., supra (2001) performed a series of antibody studies to characterize the cleavage pattern of the corneodesmosin protein. The first proteolytic cleavage results in cleavage of approximately 55 amino acids at the N terminus (since antibodies raised against amino acids 40-55 no longer bind to the cleavage product), and cleavage of approximately 58 amino acids at the C terminus (since antibodies raised against amino acids 472-486 no longer bind to the cleavage product). Accordingly, fragment a. shown in FIG. 1B consists of amino acids 1-55 in FIG. 2E (SEQ ID NO.69) and fragment b. shown in FIG. 1B consists of amino acids 472-529 in FIG. 2E (SEQ ID NO.70). The next proteolytic cleavage results in formation of fragment c by cleavage on the C side of the N-terminal glycine-rich domain, which makes fragment c in the vicinity of amino acids 55 to 200 (SEQ ID NO.71). The glycine-rich, “sticky” domains are located at amino acids 65-175 (SEQ ID NO.72) and 375-450 (SEQ ID NO.73). Although fragments a, c and d may be degraded after cleavage, it appears that fragment b may remain covalently attached to corneocytes after cleavage.

Using BLOCKS+(http://blocks.thcrc.org), a program designed to detect homologies by structure and sequence simultaneously, it can be shown that CDSN contains two of three WSC domains within its sequence (E-value=2.6e-06). (WSC is the cell wall integrity and stress response component as described by Verna et al, PNAS 94:13804-13809 (1997).) The prototypic WSC1 protein is a glycosylated and phosphorylated extracellular receptor that is an upstream regulator of the stress-activated PKC1-MAP kinase involved in the stress response of Saccharomyces cerevisiae (Lodder et al., 1999). WSC domains are present in many proteins of the extracellular matrix and are generally associated with signal transduction through the PKC pathway. This homology suggests that CDSN may be able to react to mechanical stress induced by its binding to its partners on adjacent cells by activating a signaling cascade that involves a PKC-mediated response. The truncated version of CDSN present in people with HSS does not contain one of the WSC domains and thus could possibly have lost either this signaling ability or the ability to interact with a partner that initiates signaling.

Analysis of the CDSN sequence depicted in FIG. 2B by Swiss-Prot (www.expasy.ch/cgi-bin/sprot-search-ac?Q15517) reveals a potentially cleavable signal peptide from amino acids 1 to 16 (SEQ ID NO. 16), and 9 serine (or valine) rich domains from amino acids 50-64 (SEQ ID NO. 17), 120-128 (SEQ ID NO.18), 130-144 (SEQ ID NO.19), 148-151 (SEQ ID NO.20), 179-183 (SEQ ID NO.21), 237-241 (valine rich, SEQ ID NO.22), 384-394 (SEQ ID NO.23), 414-424 (SEQ ID NO.24) and 434-439 (SEQ ID NO.25). These serine-rich domains, especially those located at the NH2 terminal end of the protein, may fold to give structural motifs similar to the glycine loops described in epidermal cytokeratins and locirin, which are proposed to exhibit adhesive properties. The valine-rich domain may have a different functionality. Each of these ten domains is underlined in FIG. 2B.

Analysis of the CDSN sequence by Prosite (www.expasy.ch/cgi-bin/scanprosite?l) reveals the functionalities in the CDSN protein shown below in Table 1.

TABLE 1

| Functionality | Position | Sequence | SEQ ID NO. |
|---|---|---|---|
| N-glycosylation | 156-159 | NGSA | SEQ ID NO. 26 |
| Protein kinase C | 41-43 | TGK | SEQ ID NO. 27 |
| phosphorylation | 64-66 | SAR | SEQ ID NO. 28 |
| | 89-91 | SFK | SEQ ID NO. 29 |
| | 166-168 | SYR | SEQ ID NO. 30 |
| | 379-381 | SSR | SEQ ID NO. 31 |
| | 424-426 | SGK | SEQ ID NO. 32 |
| | 482-484 | SIR | SEQ ID NO. 33 |
| Casein kinase II | 21-24 | TFSD | SEQ ID NO. 34 |
| phosphorylation | 34-37 | SPND | SEQ ID NO. 35 |
| | 271-274 | TSVD | SEQ ID NO. 36 |
| N-myristoylation | 54-59 | GSSSSG | SEQ ID NO. 37 |
| | 59-64 | GSSISS | SEQ ID NO. 38 |
| | 69-74 | GGGSSG | SEQ ID NO. 39 |
| | 70-75 | GGSSGS | SEQ ID NO. 40 |
| | 71-76 | GSSGSS | SEQ ID NO. 41 |
| | 74-79 | GSSSGS | SEQ ID NO. 42 |
| | 78-83 | GSSIAQ | SEQ ID NO. 43 |
| | 84-89 | GGSAGS | SEQ ID NO. 44 |
| | 85-90 | GSAGSF | SEQ ID NO. 45 |
| | 93-98 | GTGYSQ | SEQ ID NO. 46 |
| | 104-109 | GSGSSL | SEQ ID NO. 47 |
| | 111-116 | GASGSS | SEQ ID NO. 48 |
| | 119-124 | GSSSSH | SEQ ID NO. 49 |
| | 126-131 | GSSGSH | SEQ ID NO. 50 |
| | 129-134 | GSHSGS | SEQ ID NO. 51 |
| | 133-138 | GSSSSH | SEQ ID NO. 52 |
| | 155-160 | GNGSAL | SEQ ID NO. 53 |
| | 177-182 | GQSSSS | SEQ ID NO. 54 |
| | 187-192 | GVSSSG | SEQ ID NO. 55 |
| | 192-197 | GQSVSS | SEQ ID NO. 56 |
| | 260-265 | GGLPGK | SEQ ID NO. 57 |
| | 285-290 | GSSDSY | SEQ ID NO. 58 |
| | 294-299 | GMTYSK | SEQ ID NO. 59 |
| | 360-365 | GVQLCG | SEQ ID NO. 60 |
| | 365-370 | GGGSTG | SEQ ID NO. 61 |
| | 366-371 | GGSTGS | SEQ ID NO. 62 |
| | 367-372 | GSTGSK | SEQ ID NO. 63 |
| | 413-418 | GSFSSS | SEQ ID NO. 64 |
| | 433-438 | GSKSSS | SEQ ID NO. 65 |
| | 453-458 | GGPDGS | SEQ ID NO. 66 |

The sequential proteolysis of CDSN is essential for its proper functioning in hair growth. Newly discovered proteases called ADAMs (a disintegrin and metalloprotease) that are active in the extracellular matrix participate in a process known as protein ectodomain shedding which has been shown to be required for normal cellular functions during development. Many proteins with roles in development including cytokines, growth factors, receptors and adhesion molecules, are cleaved by these proteases thus releasing a protein fragment that either activates its parent and/or is available to interact with signaling molecules itself (Blobel, 2002). Although the specific proteases that cleave CDSN are not fully understood, nor is the role that the released proteolyzed fragments play in mediating CDSN function, it is postulated that CDSN activity may be mediated by modulating proteolysis.

In addition to modulating proteolysis either directly or indirectly, CDSN activity can also be modulated even more indirectly by modulating the functioning of the proteolytic fragments, or “ectodomains”, themselves. For example, fragment mimics are likely to act as modulators of hair growth. Accordingly, one aspect of the present invention is a therapeutic agent that mimics the biological activity of any one of the five CDSN proteolytic cleavage products.

Methods for modulating gene transcription and translation are well known in the art. Corneodesmosin provides an ideal target for topically administered gene therapeutics, because the affected area is at the top of the head which is easily treatable via topical application. Moreover, topical administration of gene therapies that are administered to hair follicles for treatment of alopecia are known. See, e.g., European Journal of Dermatology, 11(4):353-356 (2001). As with any gene therapy, such agents can be designed to modulate either transcription or translation, either directly via hybridization mediated interactions with the target gene, or indirectly via interactions with transcription/translation regulatory sequences. For example, a short interfering RNA (siRNA) may function as a therapeutic agent that modulates corneodesmosin mRNA translation.

As described above, corneodesmosin activity can be modulated by a variety of different approaches. As used herein, “activity associated with hair growth” refers to any role that corneodesmosin plays in proper keratinocyte or other cell function associated with hair growth, and can include, without limitation, the following: its ability to modulate cell-cell signaling; the efficiency of corneodesmosin as a protease substrate, (i.e. its ability to be proteolytically cleaved at the proper time and cleavage site); its cell adhesion properties, etc.

Nucleic Acid Mediated Applications

The present invention provides for corneodesmosin target sequence-specific probes for detecting a target nucleotide sequence associated with corneodesmosin-mediated alopecia, as well as target sequence-specific therapeutic agents that hybridize with a target nucleotide sequence associated with corneodesmosin transcription or translation. In particular, known or newly discovered abnormalities in the corneodesmosin gene can be detected using nucleic acid probe-based assay techniques. In therapeutic applications, the corneodesmosin gene sequence or a subregion thereof, or a regulatory sequence which modulates transcription or translation, can be targeted in a hybridization-mediated therapeutic application. In any event, such nucleic acid-based agents can be prepared using known methods.

More particularly, the agent can be in any suitable form. For example, the agent can comprise DNA, RNA, PNA or a derivative thereof. Alternatively, the agent can comprise both DNA and RNA or derivatives thereof. The agent can be single-stranded and be ready to be used in a hybridization analysis. Alternatively, the agent can be double-stranded and be denatured into single-stranded prior to the hybridization analysis.

The target sequence-specific agents can be produced by any suitable method. For example, the agents can be chemically synthesized (See generally, Ausubel (Ed.) Current Protocols in Molecular Biology, 2.11. Synthesis and purification of oligonucleotides, John Wiley & Sons, Inc. (2000)), isolated from a natural source, produced by recombinant methods or a combination thereof. Synthetic oligonucleotides can also be prepared by using the triester method of Matteucci et al., J. Am. Chem. Soc., 3:3185-3191 (1981). Alternatively, automated synthesis may be preferred, for example, on a Applied Biosynthesis DNA synthesizer using cyanoethyl phosphoramidite chemistry. Preferably, the agents are chemically synthesized.

Suitable bases for preparing the target sequence-specific agents of the present invention may be selected from naturally occurring nucleotide bases such as adenine, cytosine, guanine, uracil, and thymine, with the caveat that bases for preparing agents intended to be administered as therapeutics must be biologically compatible. It may also be selected from normaturally occurring or “synthetic” nucleotide bases such as 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyl uridine, dihydrouridine, 2′-O-methylpseudouridine, beta-D-galactosylqueosine, 2′-Omethylguanosine, inosine, N⁶-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N⁶-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N6-isopentenyladenosine, N-((9-.beta.-D-ribofuranosyl-2-methylthiopurine-6-yl)carbamoyl)threonine, N-((9-beta-D-ribofuranosylpurine-6-yl) N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-O-methyluridine, wybutosine, and 3-(3-amino-3-carboxypropyl) uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed in diagnostic applications. Protection from degradation can be achieved by use of a “3′-end cap” strategy by which nuclease-resistant linkages are substituted for phosphodiester linkages at the 3′ end of the oligonucleotide (Shaw et al., Nucleic Acids Res., 19:747 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem., 36:1923(1993)).

Many different chemical strategies have been employed to replace the entire phosphodiester backbone with novel linkages for diagnostic applications. Backbone analogues include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methyleneoxy (methylimino) (MOMI) linkages. Phosphorothioate and methylphosphonate-modified oligonucleotides are particularly preferred due to their availability through automated oligonucleotide synthesis. The oligonucleotide may be a “peptide nucleic acid” such as described by (Milligan et al., J. Med. Chem., 36:1923 (1993)). The only requirement is that the oligonucleotide agent should possess a sequence at least a portion of which is capable of binding to a portion of the sequence of a target DNA molecule.

The target sequence-specific agents can be of any suitable length. There is no lower or upper limits to the length of the agent, as long as the agent hybridizes to the target nucleic acid and functions effectively as a agent (e.g., facilitates detection). The agents of the present invention can be as short as 50, 40, 30, 20, 15, or 10 nucleotides, or shorter. Likewise, the agents can be as long as 20, 40, 50, 60, 75, 100 or 200 nucleotides, or longer, e.g., to the full length of the target sequence. The target sequence-specific agent is preferably short in length with a agent length of not more than 100 nucleotides, more preferably with a length between 10 and 50 nucleotides, most preferably between 20 and 40 nucleotides.

The target sequence-specific agents used in the present invention are sufficiently complementary to the target sequence to form a stable hybrid therewith. The agents need not reflect the exact complementary sequence of the target sequence, but must be sufficiently complementary to hybridize selectively with the target sequence. Non-complementary bases or longer sequences can be interspersed into the agent, provided the agent retains sufficient complementarity with the target sequence to be hybridized and to thereby form a duplex structure which can be detected.

The target sequence-specific agent need not span the entire target sequence of interest. Any subset of the target sequence that has the potential to serve as a substrate for specific binding of the agent can be targeted. Consequently, the nucleic acid agent may hybridize to as few as 8 nucleotides of the target sequence. In addition, the target sequence-specific agent should be able to hybridize with a target sequence (or portion thereof) that is at least 8 nucleotides in length under low stringency. Preferably, the agent hybridizes with a target sequence of at least 8 nucleotides under middle or high stringency.

Detecting Corneodesmosin Gene Targets

As discussed elsewhere herein, corneodesmosin gene abnormalities are implicated in alopecia. In order to diagnose the disease or propensity for the disease, the corneodesmosin abnormality associated with alopecia can be detected directly from purified DNA samples from test subjects, or amplified prior to detection using known methods to increase sensitivity.

Amplification methods suitable for use with the present methods can include, for example, polymerase chain reaction (PCR), ligase chain reaction (LCR), transcription mediated amplification (TMA) reaction, nucleic acid sequence based amplification (NASBA) reaction, and strand displacement amplification (SDA) reaction. Other methods of amplification known in the art can also be used.

PCR can be performed as according to Whelan, et al, J. Clin. Microbiol., 33(3):556-561 (1995). For example, a PCR reaction mixture can includes two specific primers, dNTP, 0.25 Units (U) of Taq polymerase, and 1×PCR Buffer. For every 25 μl PCR reaction, a 2 μl sample (e.g., isolated DNA from target organism) is added and amplified on a thermal cycler. The amplification cycle includes an initial denaturation, and up to 50 cycles of annealing, strand elongation and strand separation (denaturation).

LCR can be performed as according to Moore, et al., J. Clin. Microbiol., 36(4): 1028-1031 (1998). For example, a LCR reaction mixture can contain two pair of probes, dNTP, DNA ligase and DNA polymerase representing about 90 μl, to which is added 100 μl of isolated nucleic acid from the target organism. Amplification is performed in a thermal cycler (e.g., LCx® thermal cycler, Abbott Labs, North Chicago, Ill.).

SDA can be performed as according to Walker, et al., Nucleic Acids Res., 20(7):1691-1696 (1992). For example, an SDA reaction mixture can contain four SDA primers, dGTP, dCTP, TTP, dATPS, 150 U of Hinc II, and 5 U of exonuclease deficient E. coli DNA polymerase I. The sample mixture is heated 95° C. for 4 min to denature target DNA prior to addition of the enzymes. After addition of the two enzymes, amplification is carried out for 120 min. at 37° C. in a total volume of 50 μl. The reaction is terminated by heating for 2 min at 95° C.

NASBA can be performed as according to Heim, et al., Nucleic Acids Res., 26(9):2250-2251 (1998). For example, an NASBA reaction mixture can contain two specific primers, dNTP, NTP, 6.4 U of AMV reverse transcriptase, 0.08 U of Escherichia coli Rnase H, and 32 U of T7 RNA polymerase. The amplification is carried out for 120 min at 41° C. in a total volume of 20 μl.

TMA can be performed as according to Wylie, et al., Journal of Clinical Microbiology, 36(12):3488-3491 (1998). In TMA, nucleic acid targets are captured with magnetic beads containing specific capture primers. The beads with captured targets are washed and pelleted before adding amplification reagents, which contain amplification primers, dNTP, NTP, 2500 U of reverse transcriptase and 2500 U of T7 RNA polymerase. A 100 μl TMA reaction mixture is placed in a tube, 200 μl oil reagent is added and amplification is accomplished by incubation at 42° C. in a waterbath for one hour (“hr”).

Screening Methods

In yet another aspect of the present invention, a screening method is provided for suitable diagnostic or therapeutic agents relating to corneodesmosin-mediated alopecia. Any corneodesmosin activity can be detected in the absence or presence of a test agent and selected on the basis of its ability to modulate corneodesmosin activity in a desired manner.

Animal models for studying corneodesmosin have been described. See, e.g., Jonca, et al., J. Biol. Chem. 277(7):5024-5029. Therein, mouse fibroblasts expressing corneodesmosin were used to construct an assay for studying corneodesmosin-mediated adhesion. Such an assay is useful herein for performing initial screening of agents that are designed to modulate hair growth. In addition, animal models useful for studying hair follicles are also known (U.S. Pat. No. 6,348,348).

Therapeutic Applications

Therapeutic compositions for modulating hair growth are known in the art, as are a variety of different modes of administration and pharmaceutically acceptable carriers. For example, minoxidil which is commercially available treatment for male pattern baldness, is delivered as a 2-5% solution containing alcohol and polyethylene glycol.

General Approach to Identifying Targets

In addition to the specific corneodesmosin-related application discussed above, the present invention provides a general approach for detecting other genetic targets associated with alopecia.

a. Familial Alopecia

Genetic alopecia is easy to identify by surveying publicly available information for small populations of closely related people or individual families whose members exhibit different forms of hair loss. Such members are genotyped by studying affected and nonaffected individuals using known methods (such as single nucleotide polymorphisms, or SNPs) to locate the critical chromosomal region associated with the gene. Once the critical region is located, regions of particular interest are identified by studying the individual genes expressed in skin that are within this region.

b. Genes Expressed in Skin

There are many different publicly available sources to identify known skin genes and their chromosomal location. One such library is found at www.tigem.it/skin. These libraries of known skin-associated genes can also be generated or supplemented using, e.g., the Unigene database to identify genes specifically expressed in skin. The expression of each newly identified gene can be confirmed by semi-quantitative RT-PCR performed on RNA collected from several tissues, including skin. The expression distribution can also be analyzed by in situ hybridization at different developmental stages. Genes isolated with this strategy are mapped by bioinformatic procedures to identify candidate genes expressed in skin.

c. Locating the Abnormal Gene

By narrowing the field of suspected genes using the techniques described above, a more refined genetic study of the members of the population at issue can be carried out to pinpoint the affected gene. For example, primers are easily designed from the sequences of the known skin-associated genes which can be used to perform RT-PCR. The results of these studies should reveal the mutation or mutations that are responsible for the observed alopecia.

d. Designing Diagnostics and Therapeutics

The logical extension of this method is to use the information derived about the abnormal gene to design gene-based diagnostic agents to be able to identify the same genetic abnormality in the population at large or in members of a group carrying the abnormal gene who may be too young to be symptomatic. In this way, candidates for therapy can easily be identified and treated before disease onset. Gene-based therapeutic agents or small molecule therapies that modulate gene function can also be developed based on this method to treat such affected individuals.

### EXAMPLES

### Example 1

### Characterization of Corneodesmosin Gene Polymorphisms

As described in the “Background” section, supra, Betz, et al., Am J. Hum. Genet. 66:1997-1983 (2000), incorporated by reference, mapped a gene for hypotrichosis simplex to chromosome 6p21.3 between markers D6S276 and D6S 1607, an interval of about 15 million base pairs. This information was used to develop the experiments which are described in this example.

DNA samples were taken from members of a family, some of whom suffered from HSS, and some of whom did not. Polymorphic marker analysis was carried out and, based upon comparison of results from afflicted and non-afflicted individuals, the interval upon which the gene was found was narrowed to 11.5 megabases. Based upon information in public databases, it was estimated that about 250 genes are contained in this 11.5 megabase interval.

As HSS is associated with the skin and scalp, it was hypothesized that the gene at issue was expressed in skin. Hence, emphasis was placed upon those genes among the 250 referred to supra, that were known to be expressed.

Skin cells were taken from the family members described supra, and RT-PCR was carried out. One gene of interest was the gene referred to as “CDSN,” for corneodesmosin described by Zhou, et al Proc. Natl. Acad. Sci USA 90:9470-9474 (1993), the disclosure of which is incorporated by reference.

Zhou, et al. explain that the CDSN gene contains two exons. The first exon was amplified using primers:

GTCCAGCTC GGCATAAAGG

(SEQ ID NO:1), and

CGACCATACA GTGAGGAGCA

(SEQ ID NO:2).

The second exon was considered in four segments, each of which was amplified by primer pairs, e.g.:

AGAAAGGTGA GGGAGGAAGC

(SEQ ID NO:3), and

CCGCGGTAAG AGTTGTCATT

(SEQ ID NO:4);

CAGCAGCAGC TTTCAGTTCA

(SEQ ID NO:5), and

GAGCCTTTCA CAGGGTTCTC T

(SEQ ID NO:6);

TCCCCCAATC ACCTCTGTAG

(SEQ ID NO:7), and

CCAGAAGAGC TGGACTTGCT

(SEQ ID NO:8); and

TCAGCAGCAG CTCCAGTTC

(SEQ ID NO:9); and

AAGGAGGAAG GGGTGATAAG AG

(SEQ ID NO:10).

PCR was carried out in 96 well plates, using standard equipment. For each reaction, 50 mg of cDNA were used, in a final volume of 20 micrometers. Initial denaturation took place at 95° C. for 2 minutes, followed by 30 cycles at 95° C., 45 seconds each, to complete denaturation. For annealing, 30 cycles were carried out for 45 seconds each, at 56° C., and then thirty, sixty seconds cycles were carried out at 72° C. for extension. The final extension was carried out at 72° C., for 7 minutes.

The PCR products were purified and sequenced, using standard methods and commercially available materials.

The results indicated that there was a single change in the PCR product of individuals afflicted with the disease. Specifically, at position 657 (in FIG. 2D) from the start, a “C” was changed to “T” in afflicted individuals.

To confirm this result the amplification products were tested in a restriction enzyme assay. It was observed that the mutation, if present, created a restriction site for enzyme BfaI. The mutation occurred in the second segment of exon 2, as discussed supra. As such, 10 microliters of the PCR amplification product of normal and afflicted individuals, corresponding to exon 2, part 2 were admixed, in a 15 microliter reaction volume, with 1.5 microliters of 10×NeBuffer4, and 5 units of BfaI enzyme. The reaction volume was incubated at 37° C. Results were visualized on 2% agarose gels, stained with ethidium bromide.

The samples from subjects afflicted with HSS produced three fragments, of 528 (the WT allele), 356 and 172 base pairs, while samples from non-afflicted individuals showed a single 528 base pair band.

The sequences depicted in FIGS. 2D and 2E, respectively, are the mRNA and protein sequences of CDSN, respectively. With respect to mRNA (SEQ ID NO:14), afflicted individuals have a “T” rather than a “C” at position 657. This nucleotide is 643 basepairs downstream of the start codon, which is at nucleotides 15-17 of SEQ ID NO:14. The first 14 nucleotides are part of the untranslated region (UTR) and thus do not produce protein. This results in a stop codon. As such, in the amino acid sequence, the “Q” at position 215 is not present, nor are amino acids 216 et seq., because these are not translated in the mutated DNA. The mutation was found in all afflicted individuals studied, but in none of the 350 normal controls.

The foregoing examples thus describe nucleic acid and protein molecules associated with HSS. Specifically, the isolated nucleic acid molecule set forth at SEQ ID NO:14, with the proviso that the “C” at position 657 is “T”, is one feature of the invention. Similarly, a nucleic acid molecule consisting of nucleotides 1-657 or nucleotides 15-657, with the foregoing proviso, as well as nucleic acid molecules consisting of nucleotides 658 to the end of SEQ ID NO:14, are feature of the invention.

These nucleic acid molecules are useful both for the expression of proteins, as well as diagnostic agents. With respect to the former, as was noted supra, the mutated form of the nucleic acid molecule results in expression of a truncated, 214 amino acid protein, in contrast to the normal 529 amino acid protein. This truncated protein can be used for the generation of antibodies which are specific for the truncated form, and do not bind to the longer form of normal protein. One of ordinary skill in the art would recognize that the severity of truncation resulting from the mutation would be expected to result in conformational changes in the protein, which in turn would result in production of antibodies specific to the truncated form, rather than to linear epitopes found in the first 214 amino acids. Such antibodies, be they polyclonal, monoclonal, humanized, chimerized, or antibody fragments which retain binding specificity could be used to identify the truncated protein as a marker for HSS.

It will also be realized that antibodies specific to an epitope formed by amino acids et seq., be it linear or conformational, could be used diagnostically, in a “negative” assay. As noted, supra, in patients with HSS, the CDSN protein is only expressed in truncated form. Hence, by using an antibody directed against an epitope formed by the missing part of the protein, one can assay for the absence of normal protein, and thereby diagnose HSS in a subject.

The nucleic acid molecules of the invention can be used diagnostically as well. As noted, supra, the mutation introduced into the marker sequence results in the introduction of restriction endonuclease cleavage site. If the nucleic acid molecule is, in fact, cleaved, an oligonucleotide molecule which would normally hybridize to the wild type sequence at a portion thereof that includes nucleotide 657 of SEQ ID NO:14 will not hybridize to it any longer, since the longer molecule has been fragmented into two smaller ones. Hence an additional feature of the invention compresses oligonucleotides of a size sufficient to hybridize to the wild type transcript described herein, but which will not hybridize to the mutant form when e.g., cleaved by a restriction endonuclease. Such oligonucleotides are preferably from 17 to 100 nucleotides in length, and which are designed so as to flank position 657 of SEQ ID NO:14.

In addition to assays of the type described supra, one can assay for presence of the mutated form of the gene. Exemplary of such assays are assays based upon the use of restriction endonucleases, and determining size of nucleic acid molecules following application of the endonuclease.

The nucleic acid molecules of the invention may be utilized, e.g., in expression vectors, where the mutated form of the nucleic acid molecule, either in whole or in part, is in operable linkage with a promoter. The choice of expression vector can vary, depending upon the goals of the investigators. For example, if a glycosylated product is desired, then a yeast expression vector, or an insect cell expression vector, such as a baculovirus vector may be used. Similarly, where glycosylation is not desired, vectors suitable for inclusion in E. coli or other prokaryotic cells may be used.

The proteins resulting from the expression of the nucleic acid molecules described supra, may be used in a further aspect of the invention, which is an assay described to identify molecules which modulate the activity of either the normal CDSN protein, the truncated form, or both. Identification of such molecules can be accomplished via, e.g. combining the molecule of interest with the protein, and determining the effect of the molecule on the protein, by comparing at least one parameter to the same parameter where the protein is studied in the absence of the molecule. “Modulate” as used herein, refers to an effect on one or more properties of the protein, such as inhibiting it, agonizing it, and so forth. The artisan of ordinary skill will be familiar with the various parameters which can be studied in such assays and these will thus not be elaborated herein.

### Example 2

### CDSN Gene Polymorphisms in Bald vs. Non-Bald Males

Genetic analysis was performed on 39 individuals; 22 bald and 17 non-bald males. Polymorphisms were observed in each group. However, out of 33 SNPs, certain polymorphisms were much more common in bald individuals than in non-bald individuals for example, at position 442 S/N (G/A) and 1243 S/L (C/T). Since each of these polymorphisms is associated with an amino acid change, the likelihood that they modulate corneodesmosin activity is high.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

We claim:

1. A therapeutic composition comprising an effector of a corneodesmosin gene function associated with hair growth in a pharmaceutically acceptable carrier, wherein said effector targets a nucleic acid sequence encoding a hair growth-associated corneodesmosin activity.

2. The composition according to claim 1, wherein the effector is a small molecule, a nucleic acid or a protein.

3. The composition according to claim 1, wherein the hair growth-associated corneodesmosin activity is cell adhesion or signal transduction.

4. A therapeutic composition comprising an effector of corneodesmosin protein activity associated with hair growth in a pharmaceutically acceptable carrier, wherein said effector targets a subregion of the corneodesmosin protein exhibiting a hair growth-associated corneodesmosin activity.

5. The composition according to claim 4, wherein the effector is a small molecule, a nucleic acid or a protein.

6. The composition according to claim 4, wherein the hair growth-associated corneodesmosin activity is cell adhesion or signal transduction.

7. The composition according to claim 4, wherein the effector modulates corneodesmosin protein proteolysis.

8. The composition according to claim 4, wherein the effector is a peptide that mimics a corneodesmosin proteolysis fragment.

9. A method for modulating hair growth comprising administering to a subject an effective amount of a therapeutic agent in a pharmaceutically acceptable carrier, wherein the agent modulates at least one corneodesmosin activity associated with hair growth.

10. The method according to claim 9, wherein the therapeutic agent inhibits hair growth.

11. The method according to claim 10, wherein the therapeutic agent promotes hair growth.

12. A method for screening an agent for hair growth modulation activity comprising the steps of:
(a) incubating cells transfected with an expression construct capable of expressing corneodesmosin protein with or without the agent; and
(b) comparing activity of the corneodesmosin protein from the cells incubated with or without the agent, wherein the activity is associated with hair growth.

13. The method of claim 12, wherein the activity is cell adhesion or signal transduction.

14. A method of diagnosing corneodesmosin gene-mediated alopecia or a propensity to develop said alopecia in a subject, comprising the steps of:
(a) determining a subregion of the corneodesmosin gene suspected of having a mutation that modulates corneodesmosin activity;
(b) preparing a nucleic acid probe that binds to the subregion; and
(c) performing a hybridization assay with the nucleic acid probe to detect the presence of the mutation.

15. The method of claim 14, wherein the subregion encodes a glycine-rich domain or a signal transducing domain.

16. A method for treating alopecia comprising administering a therapeutic agent in a pharmaceutically acceptable carrier, wherein the therapeutic agent comprises corneodesmosin protein or a fragment thereof.

17. A method for treating alopecia comprising administering a nucleic acid construct to a subject comprising a promoter operably linked to a corneodesmosin gene, wherein said nucleic acid construct is capable of expressing corneodesmosin protein or fragments thereof after administration.

18. A method for identifying a genetic target associated with genetic hair loss from a population including members exhibiting hair loss comprising the steps of:
(a) identifying the population;
(b) screening the population to locate a chromosomal region associated with the hair loss;
(c) comparing the chromosomal region to known genes expressed in skin located within the region; and
(d) rescreening the population to locate a genetic abnormality in the genes expressed in skin located within the region.
